(19) United States
(12) Patent Application Publication
(10) Pub. No.: US 2010/0229627 A1
(43) Pub. Date: Sep. 16, 2010

(54) PROTECTIVE EQUIPMENT FOR PARTICULATE MATTER DETECTION DEVICE

(75) Inventors: Takashi Egami, Tokoname-City (JP); Takeshi Sakuma, Nagoya-City (JP); Masahiro Tokuda, Nagoya-City (JP); Atsuo Kondo, Okazaki-City (JP)

Correspondence Address: BURR & BROWN, PO BOX 7068, SYRACUSE, NY, 13261-7068, US

(73) Assignee: NGK Insulators, Ltd., Nagoya-City (JP)

(21) Appl. No.: 12/699,242

(22) Filed: Feb. 3, 2010

(30) Foreign Application Priority Data
Mar. 12, 2009 (JP) 2009-058854

(51) Int. Cl. G01D 11/24; G01N 33/00

(52) U.S. Cl. 73/23.31; 73/431

(57) ABSTRACT

A protective equipment 100 for the particulate matter detection device includes a bottomed cylindrical protective equipment main body 4 having a cylindrical trunk portion 1 and a bottom portion 3 which closes one end 2 of the trunk portion 1, and there are formed a gas introduction port 5 which extends through a wall of the trunk portion 1 and through which the gas can flow from the outside to the inside and a gas discharge port 6 which extends through a wall of the trunk portion 1 at a position facing the gas introduction port 5 and through which the gas can be discharged from the inside to the outside.

### BACKGROUND OF THE INVENTION

1. Field of the Invention

The present invention relates to a protective equipment for a particulate matter detection device. More particularly, it relates to a protective equipment for a particulate matter detection device, which can prevent the particulate matter detection device from being cooled by the flow of an exhaust gas in a pipe and which can prevent the particulate matter detection device from being broken down by water mixed with the exhaust gas or the like, when the particulate matter detection device is attached to the pipe of the exhaust gas or the like.

2. Description of the Related Art

A flue exhaust gas or a diesel engine exhaust gas includes a particulate matter (PM) such as soot, and has been a cause for air pollution. To remove this particulate matter, a filter (a diesel particulate filter: DPF) made of a ceramic material or the like is broadly used. The ceramic DPF can be used for a long period of time, but a defect such as cracking or melting occurs owing to heat deterioration or the like sometimes, and the particulate matter might leak though its amount is small. When such a defect occurs, it is remarkably important from the viewpoint of the prevention of the air pollution to immediately detect the occurrence of the defect and to recognize the abnormality of a device. As a method of detecting the occurrence of such a defect, a method is suggested in which a particulate matter (PM) detection device is disposed on the downstream side of the DPF (e.g., see Patent Document 1).

[Patent Document 1] JP-A-60-123761

In the invention described in Patent Document 1, the particulate matter is charged by corona discharge, and the ion current of the particulate matter is measured, thereby measuring the amount thereof. In this way, the method of charging the particulate matter to measure the ion current thereof has a problem that the ion current to charge the particulate matter is weak, whereby a large-scale detection circuit for detecting the weak ion current is required and increases cost. Moreover, when the exhaust gas has a high flow rate, the particulate matter cannot effectively be charged, thereby causing problems that the measured value of the particulate matter is smaller than the amount of the particulate matter actually included in the exhaust gas and that the error of the measurement is large.

To solve such a problem, there is suggested a particulate matter detection device made of a ceramic material, prolonged in one direction and having a through hole for detecting a particulate matter and a pair of electrodes at one end thereof and a takeout portion of a wiring line at the other end thereof (Japanese Patent Application No. 2008-246461). This particulate matter detection device is, for example, a plate-like ceramic particulate matter detection device 121 shown in FIGS. 6A and 6B. The particulate matter detection device 121 has a detecting portion 122 at one end 165 of a detection device main body 161, and the detecting portion 122 has a through hole (the through hole of the detection device main body) 162 and a pair of electrodes embedded so as to sandwich the through hole 162 therebetween. Moreover, a voltage is applied between the pair of electrodes, and the particulate matter in an exhaust gas which has flowed into the through hole 162 is electrically caused to adhere to, for example, the inner wall surface of the through hole, whereby the impedance of the inner wall surface of the through hole or the like is measured to detect the amount of the adhering particulate matter or the like. Furthermore, at an end (the other end) 166 of the main body opposite to the end thereof provided with the through hole 162, a takeout terminal 163 connected to one of the pair of electrodes is disposed, and on the surface of a position between the one end and the other end, a takeout terminal 164 connected to the other electrode of the pair of electrodes is disposed. The takeout terminals 163, 164 are portions connected to an external wiring line. FIG. 6A is a front view schematically showing the particulate matter detection device. FIG. 6B is a side view schematically showing the particulate matter detection device.

The above particulate matter detection device is disposed in a fixing member for fixing the particulate matter detection device to a pipe or the like, and the fixing member provided with the particulate matter detection device is disposed in the pipe through which the exhaust gas or the like flows, whereby the particulate matter included in the exhaust gas or the like circulating through the pipe is detected. The particulate matter detection device is remarkably useful, but room for improvement is left. For example, when the particulate matter detection device is attached to the pipe for discharging the exhaust gas from a car engine, the particulate matter detection device is cooled by the flow of the exhaust gas in the pipe, and a temperature appropriate for using the particulate matter detection device is not easily kept sometimes. In this respect, the room for improvement is left. Moreover, the particulate matter detection device heated to a high temperature is easily broken down sometimes by water mixed with the exhaust gas, and in this respect, the room for improvement is also left.

The present invention has been developed in view of such problems of the conventional technology, and there is provided a protective equipment for a particulate matter detection device, which can prevent the particulate matter detection device from being cooled by the flow of the exhaust gas in the pipe and which can prevent the particulate matter detection device from being broken down by the water mixed with the exhaust gas or the like, when the particulate matter detection device is attached to the pipe of the exhaust gas or the like.

### SUMMARY OF THE INVENTION

To achieve the above object, according to the present invention, a protective equipment for a particulate matter detection device is provided as follows.

[1] A protective equipment for a particulate matter detection device comprising: a bottomed cylindrical protective equipment main body having a cylindrical trunk portion and a bottom portion which closes one end of the trunk portion, the protective equipment main body being provided with a gas introduction port which extends through a wall of the trunk portion and through which a gas flows from the outside to the inside, and a gas discharge port which extends through the wall of the trunk portion at a position facing the gas introduction port and through which the gas is discharged from the inside to the outside.

[2] The protective equipment for the particulate matter detection device according to [1], further comprising: plate-like inlet side guide plates formed so as to cross the gas introduction port at right angles and so as to extend from at least a part of a contour portion of the gas introduction port to the inside of the trunk portion.

[3] The protective equipment for the particulate matter detection device according to [1] or [2], wherein the trunk portion includes a squeezed portion whose cross section crossing a central axis at right angles has a sectional area smaller than that of each of the other portions, and the squeezed portion is provided with the gas introduction port and the gas discharge port.

[4] The protective equipment for the particulate matter detection device according to any one of [1] to [3], further comprising: a gas introduction tube disposed outside the trunk portion so that the gas introduction port is connected to a hollow portion.

[5] The protective equipment for the particulate matter detection device according to any one of [1] to [3], wherein a through hole is formed in the bottom portion of the protective equipment main body.

[6] A tubular structure for fixing a particulate matter detection device provided with a protective equipment, comprising: a tubular first holding tube having a fixing structure portion at a first end as one end thereof, and a tubular second holding tube having one end fixed to a second end as the other end of the first holding tube so that the second holding tube is coaxial with the first holding tube, wherein at the tip of the first holding tube, the protective equipment for the particulate matter detection device according to any one of [1] to [5] is detachably attached; the particulate matter detection device made of a ceramic material, prolonged in one direction and having a detecting portion of a particulate matter at one end thereof and a takeout portion of a wiring line at the other end thereof is disposed so that the detecting portion projects to the outside from the first end of the first holding tube and so that the takeout portion of the wiring line is positioned in the second holding tube; and the particulate matter detection device is fixed to a pipe of an exhaust gas by the fixing structure portion of the first holding tube so that the detecting portion of the particulate matter detection device is positioned in the pipe of the exhaust gas.

The protective equipment for the particulate matter detection device of the present invention has a bottomed tubular shape and is provided with the gas introduction port and the gas discharge port, and hence the particulate matter detection device covered with the protective equipment can be disposed in the pipe of the exhaust gas or the like to measure the particulate matter, whereby the particulate matter detection device can be prevented from being cooled by the flow of the exhaust gas or the like in the pipe. Furthermore, water mixed with the exhaust gas or the like can be prevented from adhering to the particulate matter detection device, and hence the particulate matter detection device heated to a high temperature can be prevented from being broken down by the water.

### BRIEF DESCRIPTION OF THE DRAWINGS

FIG. 1A is a front view schematically showing one embodiment of a protective equipment for a particulate matter detection device of the present invention;

FIG. 1B is a schematic diagram showing a cross section of the protective equipment cut along the line A-A′ of FIG. 1A;

FIG. 1C is a schematic diagram showing a cross section of the protective equipment cut along the line B-B′ of FIG. 1A;

FIG. 1D is a schematic diagram showing a cross section of the protective equipment cut along the line C-C′ of FIG. 1A;

FIG. 2A is a front view schematically showing another embodiment of the protective equipment for the particulate matter detection device of the present invention;

FIG. 2B is a plan view schematically showing the embodiment of the protective equipment for the particulate matter detection device of the present invention;

FIG. 2C is a schematic diagram showing a cross section of the protective equipment cut along the line D-D′ of FIG. 2A;

FIG. 3A is a side view schematically showing still another embodiment of the protective equipment for the particulate matter detection device of the present invention;

FIG. 3B is a back view schematically showing the embodiment of the protective equipment for the particulate matter detection device of the present invention;

FIG. 3C is a front view schematically showing the embodiment of the protective equipment for the particulate matter detection device of the present invention;

FIG. 4 is a side view schematically showing one embodiment of a tubular structure for fixing the particulate matter detection device provided with the protective equipment of the present invention;

FIG. 5 is a schematic diagram showing the cross section of the embodiment of the tubular structure for fixing the particulate matter detection device provided with the protective equipment of the present invention cut along a plane parallel to a central axis direction;

FIG. 6A is a front view schematically showing the particulate matter detection device; and

FIG. 6B is a side view schematically showing the particulate matter detection device.

### DESCRIPTION OF THE PREFERRED EMBODIMENT

Next, embodiments of the present invention will be described in detail with reference to the drawings, but it should be understood that the present invention is not limited to the following embodiments and that the appropriate alteration, improvement or the like of design is added based on the ordinary knowledge of a person with ordinary skill without departing from the scope of the present invention.

(1) Protective Equipment for Particulate Matter Detection Device:

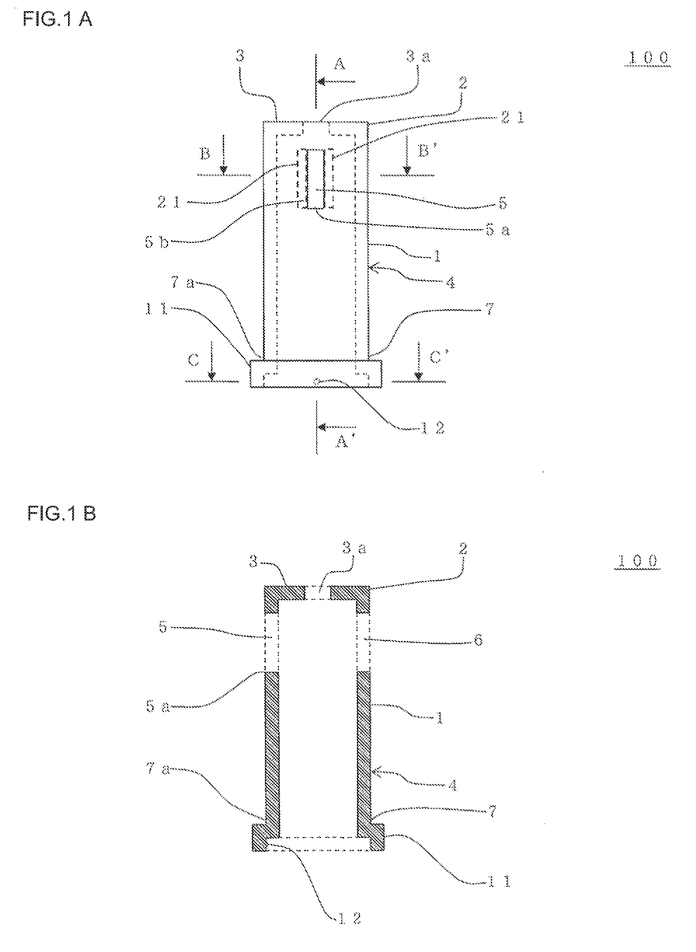

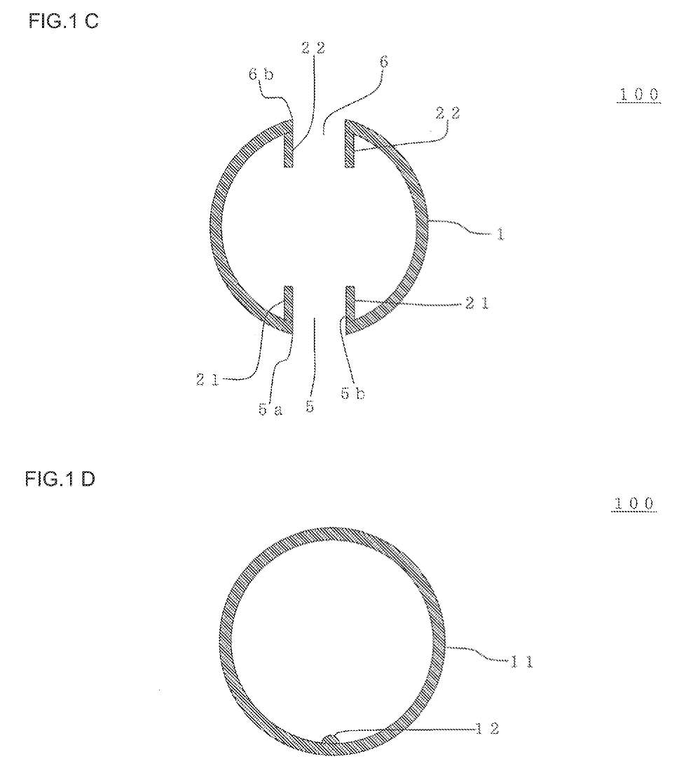

As shown in FIGS. 1A to 1D, one embodiment of a protective equipment for a particulate matter detection device of the present invention (hereinafter referred to simply as “the protective equipment” sometimes) includes a bottomed cylindrical protective equipment main body 4 having a cylindrical trunk portion 1 and a bottom portion 3 which closes one end 2 of the trunk portion 1, and is provided with a gas introduction port 5 which extends through a wall of the trunk portion 1 and through which a gas can flow from the outside to the inside, and a gas discharge port 6 which extends through the wall of the trunk portion 1 at a position facing the gas introduction port 5 and through which the gas can be discharged from the inside to the outside. Here, the wall of the trunk portion 1″ is the wall which forms the trunk portion 1. Moreover, the wall of the trunk portion 1 at the position facing the gas introduction port 5″ is the wall (the wall which forms the trunk portion 1) disposed opposite to the position where the gas introduction port 5 is formed via the central axis of the trunk portion 1. In this case, a distance between the end surface of the bottom portion 3 and the gas introduction port 5 is equal to a distance between the end surface of the bottom portion 3 and the gas discharge port 6. FIG. 1A is a front view schematically showing one embodiment of a protective equipment for a particulate matter detection device of the present invention. FIG. 1B is a schematic diagram showing a cross section of the protective equipment cut along the line A-A′ of FIG. 1A. FIG. 1C is a schematic diagram showing a cross section of the protective equipment cut along the line B-B′ of FIG. 1A. FIG. 1D is a schematic diagram showing a cross section of the protective equipment cut along the line C-C′ of FIG. 1A.

A particulate matter detection device protective equipment 100 of the present embodiment includes the bottomed cylindrical protective equipment main body 4 having the cylindrical trunk portion 1 and the bottom portion (the bottom portion of the protective equipment main body) 3 which closes the one end 2 of the trunk portion 1. The length of the trunk portion 1 is from 5 to 15 mm larger than that of a portion of the particulate matter detection device inserted into a pipe. More specifically, the length of the trunk portion 1 is preferably from 38 to 48 mm. If the length is excessively large, the trunk portion cannot enter the pipe of an exhaust gas or the like sometimes. If the length is excessively small, the particulate matter detection device does not enter the portion. Moreover, the diameter of the trunk portion 1 is preferably from 8 to 18 mm. If the diameter is excessively large, an excessively large hole needs to be formed in the pipe of the exhaust gas or the like. Furthermore, when the hole formed in the pipe is large, a member for fixing the particulate matter detection device has to be increased more than necessary. The thickness of each of the trunk portion 1 and the bottom portion 3 is preferably from 0.5 to 1.5 mm. If the thickness is excessively large, the protective equipment becomes heavy and easily drops down sometimes during use. If the thickness is excessively small, strength lowers sometimes.

The shape of the trunk portion 1 is preferably cylindrical, but the present invention is not limited to this example. The shape may be, for example, a tube-like shape with a cross section crossing the central axis thereof at right angles and having a polygonal shape such as a quadrangular shape, a pentangular shape or a hexagonal shape, an elliptic shape or the like.

The bottom portion 3 is preferably provided with a through hole (the through hole of the bottom portion of the protective equipment main body) 3a. Since the through hole 3a is formed, the water stored in the protective equipment 100 can be discharged through the through hole 3a. There is not any special restriction on the shape of the through hole 3a, and examples of the cross section of the through hole crossing the central axis of the trunk portion 1 at right angles preferably include polygonal shapes such as a quadrangular shape, a pentangular shape and a hexagonal shape, a round shape and an elliptic shape. Moreover, there is not any special restriction on the size of the through hole 3a, and, for example, the size of the cross section of the through hole crossing the central axis of the trunk portion 1 at right angles is preferably from 7 to 38.5 mm², further preferably 12.5 to 28.3 mm². If the size is smaller than 7 mm², the water is not easily discharged sometimes. If the size is larger than 38.5 mm², the particulate matter detection device is easily cooled, or the water easily adheres to the particulate matter detection device sometimes. The through hole 3a is disposed preferably in the middle portion of the bottom portion 3, further preferably at the position of the center of the gravity of the bottom portion 3. Here, the middle portion of the bottom portion 3″ has the center of the gravity at the same position as that of the bottom portion 3, an outer shape analogous to that of the bottom portion 3, and an area of 22% of the area of the bottom portion 3. If the bottom portion 3 has, for example, a round shape with a radius of 10 mm, the middle portion has its center at the same position as the center (the center of the gravity) of the bottom portion 3, and has a round shape with a radius of 4.7 mm.

The material of the protective equipment main body 4 is preferably stainless steel, and ferritic stainless steel is further preferable because it is excellent in corrosion resistance.

An opening side end 7a of the other end 7 of the protective equipment main body 4 is preferably provided with a fitting portion 11 formed to be thicker than the trunk portion 1. The fitting portion 11 is cylindrical, formed to be thicker than the trunk portion 1 so that the fitting portion is coaxial with the trunk portion 1, and disposed at the other end of the trunk portion 1. The shape of the cross section of the fitting portion 11 crossing the central axis thereof at right angles is preferably the same as (analogous to) the shape of the cross section of the trunk portion 1 crossing the central axis thereof at right angles. The fitting portion 11 of the protective equipment 100 is fitted into the tip (the tip to be inserted into the pipe) of a particulate matter detection device fitting tubular structure for fixing the particulate matter detection device to the pipe or the like, and attached to the particulate matter detection device fixing tubular structure.

The outer diameter of the fitting portion 11 is preferably 2 to 8 mm larger than the trunk portion 1. Moreover, the thickness of the fitting portion 11 is preferably from 0.5 to 2 mm. Furthermore, the length of the fitting portion 11 in the central axis direction thereof is preferably from 2 to 4 mm. If the length is excessively small, the fitting portion is not easily attached to the particulate matter detection device fixing tubular structure sometimes. If the length is excessively long, the particulate matter detection device protective equipment 100 lengthens, and is not easily inserted into the pipe sometimes. The length of the particulate matter detection device protective equipment 100 is the total of the lengths of the trunk portion 1 and the fitting portion 11. Moreover, the material of the fitting portion 11 is preferably the same as that of the protective equipment main body 4.

On the inner side of the fitting portion 11, as shown in FIGS. 1A, 1B and 1D, a projection 12 is preferably formed. The end of the particulate matter detection device fixing tubular structure on a side where the particulate matter detection device projects is provided with a concave portion which can be fitted into the projection 12, whereby when the protective equipment 100 is attached to the particulate matter detection device fixing tubular structure, the projection 12 can be fitted into the concave portion to easily attach the protective equipment 100 to the particulate matter detection device fixing tubular structure. The size of the projection 12 can appropriately be determined in accordance with the size, mass and the like of the protective equipment 100, and the projection preferably has, for example, a columnar shape having a height of 1 to 3 mm and an outer diameter of 1 to 3 mm. The material of the projection 12 is preferably the same as that of the protective equipment main body 4.

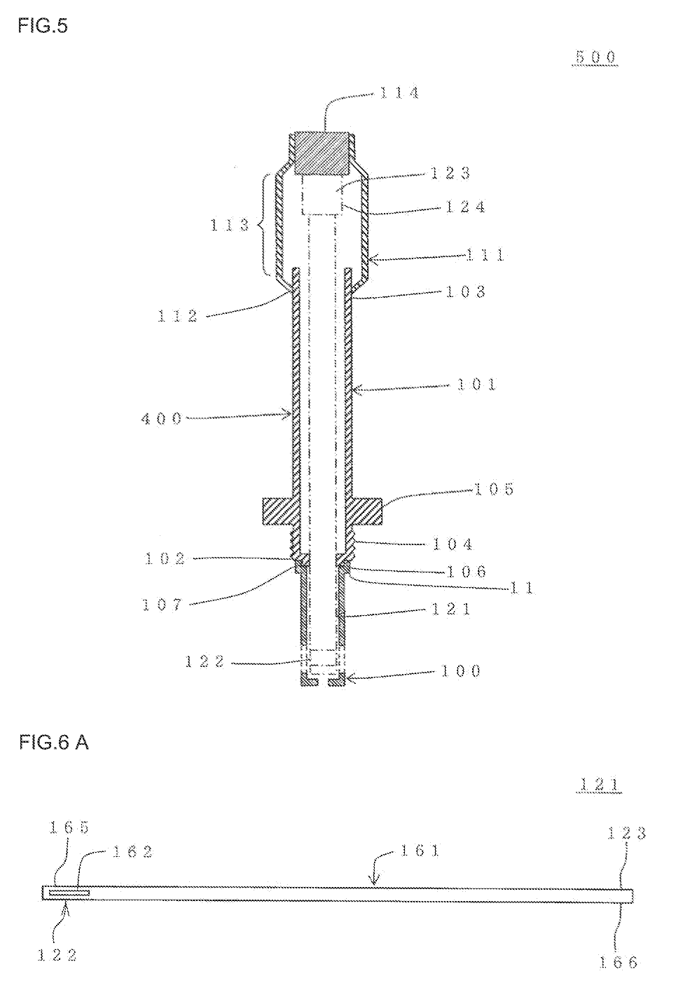

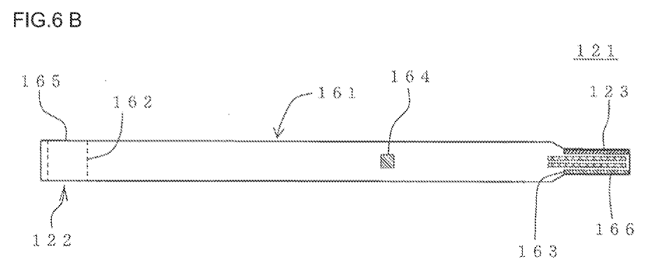

The particulate matter detection device protective equipment 100 of the present embodiment is provided with the gas introduction port 5 which extends through the wall of the trunk portion 1 and through which the gas can flow from the outside to the inside and the gas discharge port 6 which extends through the wall of the trunk portion 1 at a position facing the gas introduction port 5 and through which the gas can be discharged from the inside to the outside. The size of the gas introduction port 5 (the area of an opening 5a) is preferably as small as possible in a range in which the exhaust gas or the like can sufficiently be introduced into the portion of the particulate matter detection device for detecting the particulate matter. In a case where as shown in FIGS. 6A and 6B, the particulate matter detection device is a particulate matter detection device 121 made of a ceramic material, prolonged in one direction and having a particulate matter detecting portion 122 at one end 165 thereof and a takeout portion of a wiring line at the other end 166 thereof, the size of the gas introduction port 5 (the area of the opening 5a) is preferably from 100 to 300%, further preferably from 150 to 200% of the size (the area) of the opening of a through hole (the through hole of a detection device main body) 162 of the detecting portion 122. Specifically, the size is from 7.5 to 22.5 mm², preferably from 11.3 to 15.0 mm². If the size of the gas introduction port 5 is excessively small, the exhaust gas or the like cannot easily sufficiently be supplied to the particulate matter detection device sometimes. Moreover, if the size of the gas introduction port 5 is excessively large, the particulate matter detection device is easily cooled sometimes, and water easily enters the protective equipment 100 sometimes. When the opening 5a of the gas introduction port 5 has, for example, a rectangular shape, the length of the opening 5a of the gas introduction port 5 in the central axis direction of the trunk portion 1 is preferably from 5 to 7.5 mm, further preferably from 5.5 to 7.0 mm. The size and shape of the opening of the gas discharge port 6 are preferably the same as those of the opening of the gas introduction port 5. Moreover, the shapes of the openings of the gas introduction port 5 and the gas discharge port 6 are preferably the same as (analogous to) the shape of the opening of the through hole 162 of the particulate matter detection device 121. The shapes of the openings of the gas introduction port 5 and the gas discharge port 6 are preferably, for example, rectangular.

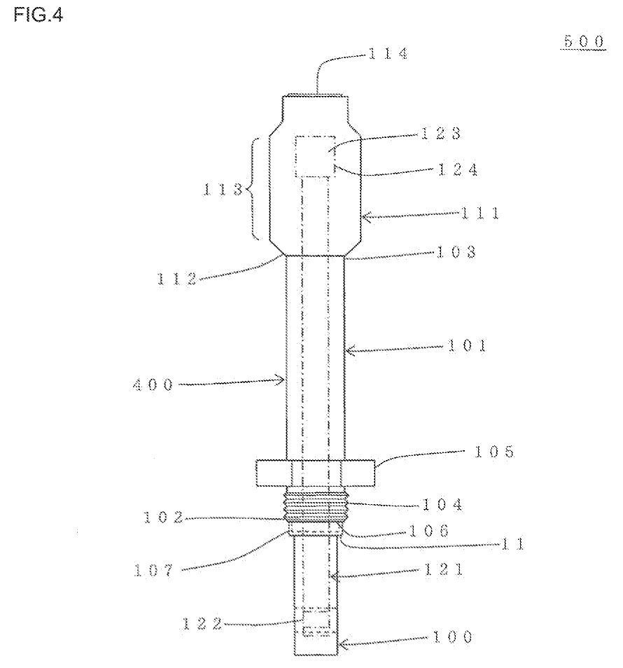

As to the arrangement of the gas introduction port 5 in the central axis direction of the trunk portion 1, as shown in FIGS. 4 and 5, when the particulate matter detection device is attached to the tubular structure for fixing the particulate matter detection device and the protective equipment 100 is attached to the tubular structure for fixing the particulate matter detection device, the opening (see FIG. 6A) of the through hole 162 of the detecting portion 122 of the particulate matter detection device is preferably positioned so as to overlap with the opening 5a of the gas introduction port 5. Since the opening of the through hole 162 overlaps with the opening 5a of the gas introduction port 5, the exhaust gas or the like can efficiently be introduced into the detecting portion (the through hole) of the particulate matter detection device. “The opening of the through hole 162 overlaps with the gas introduction port 5” indicates that if the opening 5a of the gas introduction port 5 is moved into the trunk portion 1 (the inside) in a direction crossing the opening 5a of the gas introduction port 5 at right angles and moved to the particulate matter detection device 121, at least a part of the opening 5a of the gas introduction port 5 overlaps with the opening of the through hole 162. This is based on the assumption that when the protective equipment 100 is attached to the tubular structure for fixing the particulate matter detection device, the protective equipment 100 is attached to the tubular structure for fixing the particulate matter detection device so that a direction from the central axis of the protective equipment 100 to the center of the opening of the through hole 162 becomes the same direction as that from the central axis of the protective equipment 100 to the center of the opening 5a of the gas introduction port 5 in the cross section of the protective equipment 100 crossing the central axis at right angles. Moreover, if the opening 5a of the gas introduction port 5 is moved into the trunk portion 1 in the direction crossing the opening 5a of the gas introduction port 5 at right angles and moved to the particulate matter detection device 121, the opening 5a of the gas introduction port 5 further preferably completely includes the opening of the through hole (the through hole of the detection device main body) 162. In consequence, the exhaust gas or the like can further efficiently be introduced into the detecting portion (the through hole) of the particulate matter detection device.

A distance between the end surface of the bottom portion 3 and the gas introduction port 5 is specifically preferably from 3 to 5 mm. If the distance is smaller than 3 mm, the heat of the particulate matter detection device is taken sometimes. If the distance is longer than 5 mm, the flow of the exhaust gas is obstructed sometimes.

As shown in FIGS. 1A and 1C, the particulate matter detection device protective equipment 100 of the present embodiment further includes plate-like inlet side guide plates 21 formed so as to cross the gas introduction port 5 at right angles and so as to extend from at least a part of a contour portion 5b of the gas introduction port 5 to the inside of the trunk portion 1. The contour portion 5b of the gas introduction port 5″ is a portion of a wall forming the contour of the opening of the gas introduction port 5, inside of the trunk portion 1. Since the inlet side guide plates 21 are disposed, the exhaust gas or the like introduced through the gas introduction port 5 can efficiently be introduced into the through hole of the particulate matter detection device. When the opening 5a of the gas introduction port 5 is rectangular as shown in FIG. 1A, the inlet side guide plates 21 are preferably disposed at positions corresponding to two long sides of the rectangular opening 5a of the gas introduction port 5. In this case, the length of each of the inlet side guide plates 21 in the central axis direction of the trunk portion 1 is preferably equal to that of the opening 5a in the central axis direction of the trunk portion 1. There is not any special restriction on the thickness of the inlet side guide plate 21, but the thickness is preferably from 0.2 to 1.0 mm, further preferably 0.3 to 0.8 mm. If the thickness is excessively small, strength lowers sometimes. If the thickness is excessively large, the heat is taken sometimes. The inlet side guide plates 21 are preferably disposed so as to cross the opening 5a of the gas introduction port 5 at right angles. The length of the inlet side guide plate 21 in the direction from the opening 5a of the gas introduction port 5 to the inside of the trunk portion 1 is preferably from 1 to 5 mm, further preferably from 2 to 3 mm. If the length is excessively small, an effect of guiding the exhaust gas or the like into the particulate matter detection device lowers sometimes. If the length is excessively large, the exhaust gas strikes (collides with) the particulate matter detection device, and breaks down the particulate matter detection device sometimes. There is not any special restriction on the shape of the inlet side guide plate 21, but the inlet side guide plate is preferably rectangular. Moreover, the inlet side guide plates 21 may be formed into a cylindrical shape along the whole contour of the opening 5a of the gas introduction port 5. Furthermore, as shown in FIG. 1C, the particulate matter detection device protective equipment 100 of the present embodiment preferably further includes plate-like outlet side guide plates 22 formed so as to cross the gas discharge port 6 at right angles and so as to extend from at least a part of a contour portion 6b of the gas discharge port 6 to the inside of the trunk portion 1. Since the outlet side guide plates 22 are disposed, the exhaust gas or the like discharged from the particulate matter detection device can be discharged from the protective equipment 100 with a small load (smoothly). Conditions such as the structure and arrangement of the outlet side guide plates 22 are preferably similar to those of the inlet side guide plates 21. The material of the inlet side guide plates 21 and the outlet side guide plates 22 is preferably the same as that of the protective equipment main body 4.

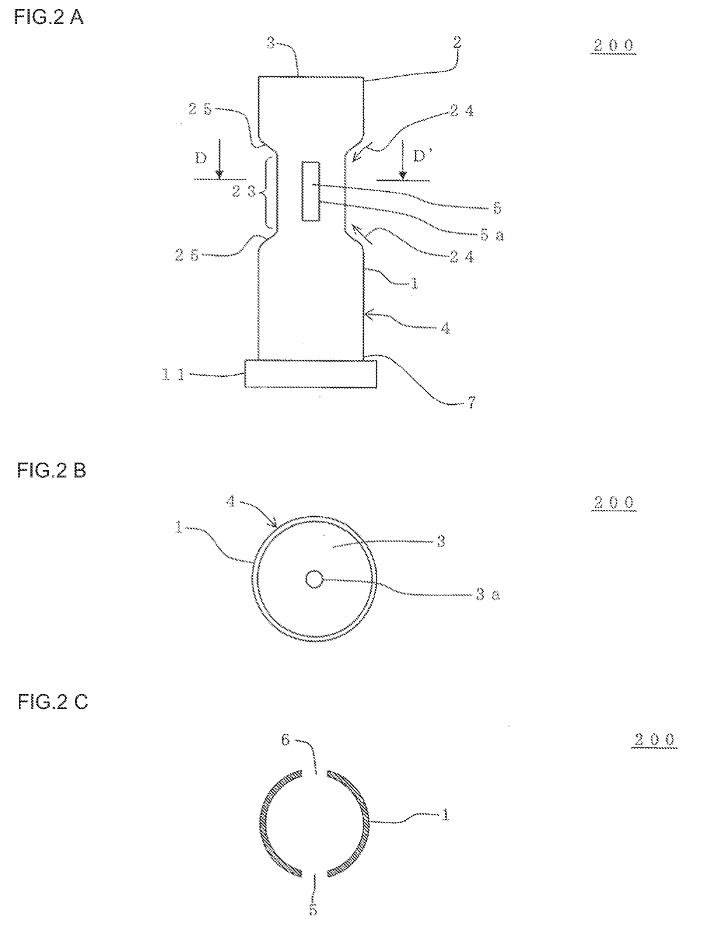

In another embodiment of the protective equipment for the particulate matter detection device of the present invention, as shown in FIGS. 2A, 2B and 2C, a trunk portion 1 has a cylindrical squeezed portion 23, the sectional area of the cross section of the squeezed portion crossing the central axis at right angles is smaller than that of each of the other portions, and the squeezed portion 23 is provided with a gas introduction port 5 and a gas discharge port 6. In a particulate matter detection device protective equipment 200 of the present embodiment, the trunk portion 1 is provided with the squeezed portion 23 in this manner, and the squeezed portion 23 is provided with the gas introduction port 5 and the gas discharge port 6. Therefore, when an exhaust gas flows so as to gather in the squeezed portion 23 (as shown by arrows of “exhaust gas flows 24” of FIG. 2A, the flow of the exhaust gas flowing toward the squeezed portion 23 is formed on the surface of the trunk portion 1 around the squeezed portion 23), the exhaust gas or the like is easily collected in the gas introduction port 5, and can more efficiently be introduced into the protective equipment 200. FIG. 2A is a front view schematically showing the other embodiment of the protective equipment for the particulate matter detection device of the present invention. FIG. 2B is a plan view schematically showing the embodiment of the protective equipment for the particulate matter detection device of the present invention. FIG. 2C is a schematic diagram showing a cross section of the protective equipment cut along the line D-D′ of FIG. 2A.

The diameter of the squeezed portion 23 is preferably such a diameter that the particulate matter detection device can be inserted into the squeezed portion. Moreover, the diameter is preferably from 77 to 85%, further preferably from 79 to 81% of the thickest cylindrical portion of the trunk portion 1. If the diameter is smaller than 77% of that of the trunk portion 1, the particulate matter detection device is not easily inserted sometimes. If the diameter is larger than 85% of that of the trunk portion 1, an effect of collecting the exhaust gas in the squeezed portion 23 (the gas introduction port 5) lowers sometimes. The diameter of the thickest cylindrical portion of the trunk portion 1″ is the diameter of the trunk portion 1 in a state in which the squeezed portion 23 is not formed, and the diameter of the end of the trunk portion 1 after provided with the squeezed portion 23 (e.g., the end on a side where a bottom portion 3 is disposed). Moreover, as shown in FIG. 2A, connecting portions 25 which connect the thickest cylindrical portion of the trunk portion 1 to the squeezed portion 23 preferably tilt by preferably 25 to 35°, further preferably 29 to 31° from the central axis of the trunk portion 1. It is to be noted that an intersecting portion between the connecting portion 25 positioned on the one end 2 side of the trunk portion 1 and the side surface of the trunk portion 1 is disposed on the one end 2 side of the trunk portion 1 from an intersecting portion between the connecting portion 25 positioned on the one end 2 side of the trunk portion 1 and the squeezed portion 23. Moreover, an intersecting portion between the connecting portion 25 positioned on the other end 7 side of the trunk portion 1 and the side surface of the trunk portion 1 is disposed on the other end 7 side of the trunk portion 1 from an intersecting portion between the connecting portion 25 positioned on the other end 7 side of the trunk portion 1 and the squeezed portion 23.

The sectional area of the cross section of the squeezed portion 23 crossing the central axis at right angles is preferably from 70 to 78%, further preferably from 73 to 75% of the sectional area of the cross section of the thickest cylindrical portion of the trunk portion 1 crossing the central axis at right angles. If the area is smaller than 70%, the particulate matter detection device is not easily inserted sometimes. If the area is larger than 78%, the effect of collecting the exhaust gas in the squeezed portion 23 (the gas introduction port 5) lowers sometimes.

A distance between the end surface of the bottom portion 3 and the squeezed portion 23 is preferably from 7 to 11 mm, further preferably 9 to 9.5 mm. In consequence, the gas introduction port 5 and the gas discharge port 6 can be formed at preferable positions. Moreover, the length of the squeezed portion 23 in the central axis direction is preferably from 5 to 10 mm, further preferably from 6 to 6.5 mm. If the length of the squeezed portion 23 in the central axis direction is excessively large, the effect of collecting the exhaust gas in the gas introduction port 5 lowers sometimes. If the length of the squeezed portion 23 in the central axis direction is excessively small, the gas introduction port 5 and the gas discharge port 6 having preferable shapes and sizes may not easily formed. A constitution and conditions of the particulate matter detection device protective equipment 200 of the present embodiment are preferably similar to those of the particulate matter detection device protective equipment 100 of the present invention except that the squeezed portion 23 is formed and that the squeezed portion 23 is provided with the gas introduction port 5 and the gas discharge port 6.

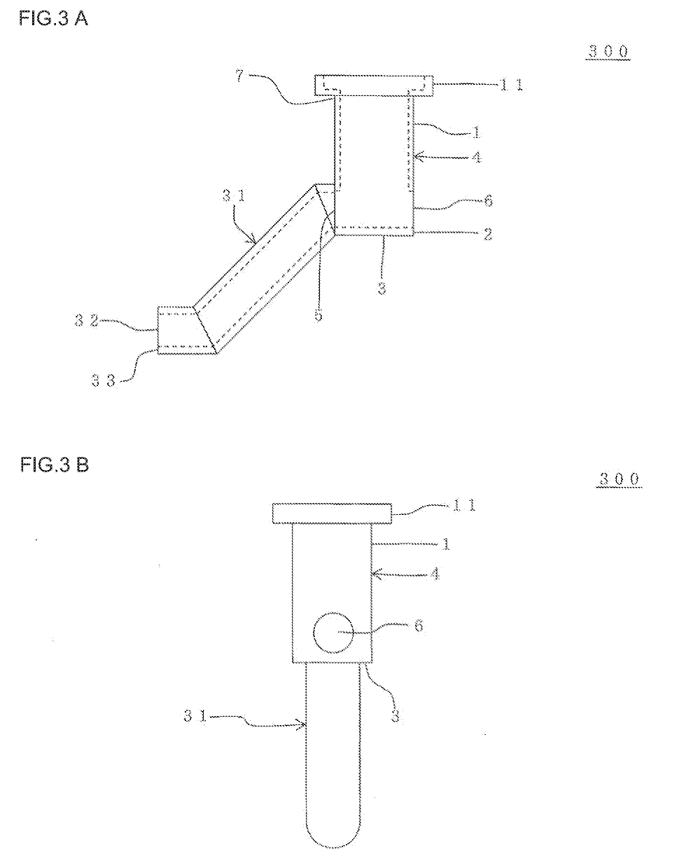

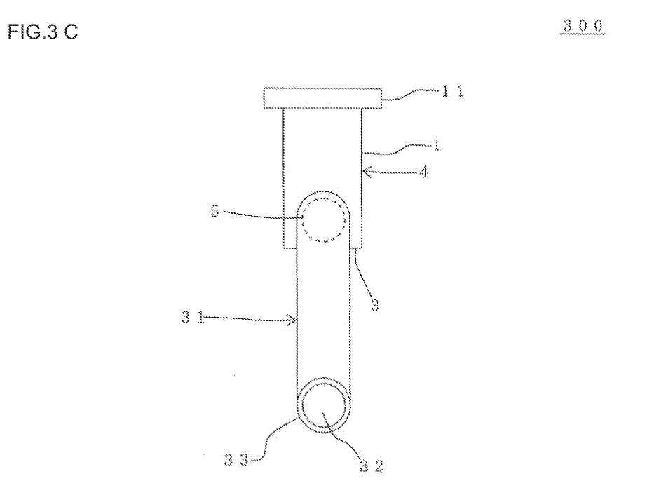

As shown in FIGS. 3A to 3C, still another embodiment of the protective equipment for the particulate matter detection device of the present invention further includes a gas introduction tube 31 disposed outside a trunk portion 1 so that a gas introduction port 5 is connected to a hollow portion 32. The gas introduction tube 31 is cylindrical and is disposed so as to cover the gas introduction port 5. In a particulate matter detection device protective equipment 300 of the present embodiment, since the gas introduction tube 31 is connected to the gas introduction port 5 of the trunk portion 1 in this manner, the particulate matter detection device can be prevented from being cooled, and can be prevented from being broken down owing to water. Furthermore, a gas inlet 33 of the gas introduction tube 31 can be disposed at a desired position in a pipe of an exhaust gas (at the desired position in the cross section of the pipe crossing a gas flow direction at right angles), whereby the exhaust gas can more efficiently be collected from a desired position such as the position in the pipe where the exhaust gas flows most. Furthermore, when the gas inlet 33 is disposed at the desired position in the pipe, the exhaust gas in the pipe can efficiently be collected, whereby the detecting portion (the through hole) of the particulate matter detection device does not have to be disposed at the above desired position in the pipe, and hence the particulate matter detection device can be shortened. In a case where the particulate matter detection device is shortened, as compared to a case where the device is prolonged, the strength of the particulate matter detection device improves, and the breakdown or the like thereof can more effectively be prevented. FIG. 3A is a side view schematically showing the other embodiment of the protective equipment for the particulate matter detection device of the present invention. FIG. 3B is a back view schematically showing the embodiment of the protective equipment for the particulate matter detection device of the present invention. FIG. 3C is a front view schematically showing the embodiment of the protective equipment for the particulate matter detection device of the present invention.

The diameter of the gas introduction tube 31 is preferably smaller than that of the trunk portion 1. Moreover, the size (the area) of a portion where the gas introduction tube 31 is connected to the trunk portion 1 is preferably larger than that of the opening of the gas introduction port 5, and the opening of the gas introduction port 5 is preferably disposed on the inner side of the portion where the gas introduction tube 31 is connected to the trunk portion 1. The diameter of the gas introduction tube 31 is specifically preferably from 5 to 10 mm, further preferably from 8 to 8.5 mm. The length of the gas introduction tube 31 is such a length that the inlet 33 of the gas introduction tube 31 is disposed at a desired position in the pipe, when the particulate matter detection device protective equipment 300 of the present embodiment is attached to the particulate matter detection device fixing tubular structure to which the particulate matter detection device is fixed, and connected to the pipe of the exhaust gas. The thickness (the wall thickness) of the gas introduction tube 31 is preferably from 0.5 to 2 mm, further preferably from 0.8 to 1.2 mm. If the thickness is excessively small, strength lowers sometimes. If the thickness is excessively large, the size of the hollow portion decreases, and the exhaust gas is not easily introduced. The material of the gas introduction tube 31 is preferably the same as that of a protective equipment main body 4. An angle between the gas introduction tube 31 and the trunk portion 1 is preferably from 35 to 55°, further preferably 43 to 47°. If the angle is smaller than 35°, the exhaust gas is not easily introduced into the protective equipment 300 sometimes. If the angle is larger than 55°, the gas inlet 33 of the gas introduction tube 31 is not easily disposed at the desired position in the pipe. It is to be noted that the angle between the gas introduction tube 31 and the trunk portion 1″ is an angle between a direction from the end of the gas introduction tube 31 disposed at the trunk portion 1 to the gas inlet 33 and a direction from the other end 7 of the trunk portion 1 to one end 2 thereof.

In the particulate matter detection device protective equipment 300 of the present embodiment, the shape of the gas introduction port 5 or the shape of a gas discharge port 6 has a round cross section thereof crossing a gas circulating direction at right angles.

A constitution and conditions of the particulate matter detection device protective equipment 300 of the present embodiment are preferably similar to those of the particulate matter detection device protective equipment 100 of the present invention except that the gas introduction tube 31 is disposed and that the gas introduction port 5 and the gas discharge port 6 have a round sectional shape.

(2) Manufacturing Method of Protective Equipment for Particulate Matter Detection Device:

A manufacturing method of the particulate matter detection device protective equipment 100 shown in FIGS. 1A to 1D will be described. Austenitic stainless steel is pressed to prepare a trunk portion. A fitting portion has a projection, and hence is prepared by cutting. The gas introduction port 5 and the gas discharge port 6 are cut simultaneously with the preparation of the trunk portion by the pressing, The inlet side guide plates 21 and the outlet side guide plates 22 are cut from a similar material, and welded to the gas introduction port 5 and the gas discharge port 6, respectively, by laser welding or the like. In this case, the trunk portion is preferably prepared so that any burr, welding sputter or the like is not present in the inside and outside of the trunk portion. The trunk portion is preferably bonded to the fitting portion by laser welding or the like to prepare the particulate matter detection device protective equipment 100.

Moreover, a manufacturing method of the particulate matter detection device protective equipment 200 shown in FIGS. 2A to 2C will be described. A trunk portion is prepared by a press in the same manner as in the particulate matter detection device protective equipment 100. In this case, the squeezed portion 23, the gas introduction port 5 and the gas discharge port 6 of the trunk portion are simultaneously formed. A fitting portion is prepared by cutting, and the trunk portion is preferably bonded to the fitting portion by laser welding or the like to prepare the particulate matter detection device protective equipment 200.

Furthermore, a manufacturing method of the particulate matter detection device protective equipment 300 shown in FIGS. 3A to 3C will be described. A trunk portion is prepared by a press in the same manner as in the particulate matter detection device protective equipment 100. When a trunk portion is prepared by a press, a gas introduction tube attaching portion as a portion to be attached to the gas introduction tube 31 and the gas discharge port 6 are cut by the press. The gas introduction tube is cut from the same material (the same material as that of the trunk portion) having a desired dimension, and prepared by laser welding or the like. Afterward, the gas introduction tube is bonded to the trunk portion by laser welding or the like, and the fitting portion is preferably bonded by laser welding or the like to prepare the particulate matter detection device protective equipment 300.

(3) Tubular Structure for fixing Particulate Matter Detection Device Provided with Protective Equipment:

(3-1) Tubular Structure for Fixing Particulate Matter Detection Device:

First, a particulate matter detection device fixing tubular structure of a tubular structure for fixing a particulate matter detection device provided with a protective equipment will be described. As shown in FIGS. 4 and 5, a particulate matter detection device fixing tubular structure (hereinafter referred to simply as “the tubular structure” sometimes) 400 of one embodiment of the tubular structure for fixing the particulate matter detection device provided with the protective equipment of the present invention (a tubular structure 500 for fixing the particulate matter detection device provided with the protective equipment) includes a tubular first holding tube 101 having a fixing structure portion 104 at a first end 102 as one end thereof, and a tubular second holding tube 111 having one end 112 fixed to a second end 103 as the other end of the first holding tube 101 so that the second holding tube is coaxial with the first holding tube 101. Moreover, in the particulate matter detection device fixing tubular structure (the tubular structure) 400, a particulate matter detection device 121 made of a ceramic material, prolonged in one direction and having a detecting portion 122 of a particulate matter at one end thereof and a takeout portion 123 of a wiring line at the other end thereof is disposed so that the detecting portion 122 projects to the outside from the first end 102 of the first holding tube 101 and so that the takeout portion 123 of the wiring line is positioned in the second holding tube 111, and the particulate matter detection device is fixed to a pipe of an exhaust gas by the fixing structure portion 104 of the first holding tube 101 so that the detecting portion 122 of the particulate matter detection device 121 is positioned in the pipe of the exhaust gas. It is to be noted that the tubular structure for fixing the particulate matter detection device is not limited to the above structure as long as the particulate matter detection device can be inserted into and fixed to the tubular structure while the end of the particulate matter detection device provided with the detecting portion projects to the outside and as long as the tubular structure provided with the particulate matter detection device can be fixed to the pipe of the exhaust gas. FIG. 4 is a side view schematically showing the embodiment of the tubular structure for fixing the particulate matter detection device provided with the protective equipment of the present invention. FIG. 5 is a schematic diagram showing a cross section of the embodiment of the tubular structure for fixing the particulate matter detection device provided with the protective equipment of the present invention cut along a plane parallel to a central axis direction.

The first holding tube 101 of the tubular structure 400 is a cylindrical member having the fixing structure portion 104 at the first end 102 as one end thereof. The first holding tube 101 is a cylindrical single tube, and hence has satisfactory heat release properties, whereby the inside of the tube can be prevented from becoming a high temperature. The first holding tube 101 is preferably cylindrical in this manner, and the cross section of the tube crossing the central axis at right angles may have a polygonal shape such as a hexagonal shape or an octagonal shape, or an elliptic shape. A distance (a length of an externally exposed portion) of the first holding tube 101 between the tip of the first end 102 and a portion bonded to the second holding tube 111 is preferably from 40 to 70 mm, further preferably 50 to 60 mm. If the distance is shorter than 40 mm, a distance between the pipe and the second holding tube 111 shortens, and the takeout portion 123 of the particulate matter detection device 121 disposed in the second holding tube 111 becomes the high temperature sometimes when the tubular structure 400 is fixed to the pipe in use. If the distance is longer than 70 mm, the whole tubular structure 400 lengthens, and is not easily used in a narrow place sometimes as in a case where the device is fixed to the pipe of the exhaust gas from a car engine. It is to be noted that in FIGS. 4 and 5, the takeout portion 123 of the particulate matter detection device 121 is hidden in a contact member 124.

The outer diameter of the cross section of the first holding tube 101 crossing the central axis thereof at right angles is preferably from 10 to 20 mm, further preferably from 12 to 16 mm. If the diameter is smaller than 10 mm, the particulate matter detection device does not easily enter the tube, and heat release becomes insufficient. If the diameter is larger than 20 mm, the device cannot easily be used in the small place as in the case where the device is fixed to the pipe of the exhaust gas from the car engine. The thickness of a wall (the wall thickness) of the first holding tube 101 is preferably from 0.3 to 1.5 mm, further preferably from 0.5 to 1.0 mm. If the thickness is smaller than 0.3 mm, strength lowers sometimes. If the thickness is larger than 1.5 mm, the heat release becomes insufficient sometimes.

The tip portion of the first end 102 of the first holding tube 101 has a bottom portion (the bottom portion of the tubular structure) 106 provided with a hole through which the particulate matter detection device 121 passes. The through hole (the hole of the bottom portion of the tubular structure) formed in the bottom portion (the bottom portion of the tubular structure) preferably has such a size that when the particulate matter detection device 121 is passed through the hole, a gap of 0.1 to 0.5 mm is formed between the periphery of the hole and the particulate matter detection device 121. Therefore, the tip portion of the first end 102 of the first holding tube 101 is closed with the bottom portion (the bottom portion of the tubular structure) 106 and the particulate matter detection device 121, which prevents the exhaust gas in the pipe from entering the tip portion of the first end 102 of the first holding tube 101.

In the tubular structure 400, the fixing structure portion 104 formed at the first end 102 of the first holding tube 101 has a thread structure (an external thread). That is, the tubular structure 400 is provided with the external thread as the fixing structure portion 104 at the first end 102 of the first holding tube 101. In consequence, forming of an internal thread on a pipe side enables the fixing of the tubular structure 400 to the pipe by thread fastening. Moreover, the tubular structure 400 has a flange portion 105 having a hexagonal cross section crossing the central axis at right angles so that the structure is tightened by a spanner, a monkey wrench or the like when thread-fastened to the pipe. In consequence, when the tubular structure 400 is fixed to the pipe by the thread fastening, the flange portion 105 is held and turned by the tip of the spanner, the monkey wrench or the like, thereby enabling the thread fastening.

Moreover, the first holding tube 101 may be constituted of the first end 102 and a trunk portion which is independent of the first end 102 and which is a portion other than the first end 102. Furthermore, the first end 102 may be formed of the integrally formed fixing structure portion 104 and flange portion 105. Additionally, in this case, the integrally formed fixing structure portion 104 and flange portion 105 are preferably attached to the trunk portion independently of the trunk portion so that they can be rotated at the end of the trunk portion. Moreover, the integrally formed fixing structure portion 104 and flange portion 105 preferably have a ring-shaped stopper portion near the first end 102 of the trunk portion of the first holding tube 101 so that they do not move toward the second end of the first holding tube 101.

There is not any special restriction on the material of the first holding tube 101, but, for example, inexpensive stainless steel having a high strength is preferable. As the type of stainless steel, austenite-based steel or the like is preferable.

The second holding tube 111 of the tubular structure 400 is a cylindrical single tube having the one end 112 fixed to the second end 103 as the other end of the first holding tube 101 so that the second holding tube is coaxial with the first holding tube 101. The second holding tube 111 is the cylindrical single tube, and hence has satisfactory heat release properties, whereby the inside of the tube can be prevented from becoming a high temperature. The second holding tube 111 is preferably cylindrical in this manner, and the cross section of the tube crossing the central axis at right angles may have a polygonal shape such as a hexagonal shape or an octagonal shape, or an elliptic shape. The length of the second holding tube 111 in the central axis direction is preferably from 30 to 60 mm, further preferably 40 to 50 mm. If the length is shorter than 30 mm, the takeout portion 123 of the tubular structure 400 cannot be received in the tube sometimes. If the length is longer than 60 mm, the whole tubular structure 400 lengthens, and cannot easily be used in the narrow place sometimes as in the case where the detection device is fixed to the pipe of the exhaust gas from the car engine.

The outer diameter of the cross section of the second holding tube 111 crossing the central axis at right angles is preferably from 15 to 25 mm, further preferably from 17 to 20 mm. If the diameter is smaller than 15 mm, the particulate matter detection device does not easily enter the tube, or heat release becomes insufficient sometimes. If the diameter is larger than 25 mm, the structure cannot easily be used in the narrow place sometimes as in the case where the device is fixed to the pipe of the exhaust gas from the car engine. The outer diameter of the cross section of the second holding tube 111 crossing the central axis at right angles is the outer diameter of a middle portion excluding both end portions having decreased diameters. The thickness of a wall of the second holding tube 111 (the wall thickness) is preferably from 0.3 to 1.5 mm, further preferably 0.5 to 1.0 mm. If the thickness is smaller than 0.3 mm, strength lowers sometimes. If the thickness is larger than 1.5 mm, the heat release becomes insufficient sometimes.

The end (the one end 112) of the second holding tube 111 connected to the first holding tube 101 and the opposite end thereof are formed to be thin, and a middle portion 113 of the tube is formed to be thick. Moreover, the diameter of the middle portion 113 (the diameter (the outer diameter) of the cross section of the portion crossing the central axis at right angles) is larger than that of the first holding tube 101. The diameter of the middle portion 113 of the second holding tube 111 is increased in this manner, whereby the takeout portion 123 of the particulate matter detection device 121 can easily be positioned in the second holding tube 111. When the takeout portion 123 of the particulate matter detection device 121 is connected to an external wiring line, the takeout portion 123 is brought into contact with the wiring line, pressed from the outside by the contact member 124, thus held and connected, whereby the volume of the takeout portion becomes larger than that of each of the other portions of the particulate matter detection device 121. Therefore, the middle portion 113 of the second holding tube 111 is preferably thickened.

There is not any special restriction on the material of the second holding tube 111, but, for example, inexpensive stainless steel having a high strength is preferable. As the type of stainless steel, austenite-based steel or the like is preferable.

The end (the other end) of the second holding tube 111 which is not connected to the first holding tube 101 is opened, and the opening is preferably closed with a plug 114 made of a rubber. Moreover, the rubber plug 114 is preferably provided with a through hole through which the external wiring line to be electrically connected to the takeout portion 123 of the particulate matter detection device 121 is passed. The rubber material of the plug preferably has a high heat resistance, and examples of the material include a silicon rubber.

The first holding tube 101 is preferably firmly connected to the second holding tube 111. They are preferably connected by, for example, laser welding, tungsten inert gas (Tig) welding or the like. Moreover, a distance between the portion of the first holding tube 101 connected to the second holding tube 111 and the tip of the second end 103 inserted into the second holding tube 111 is preferably from 5 to 15 mm, further preferably from 6 to 8 mm. If the distance is shorter than 5 mm, the strength runs short sometimes. If the distance is longer than 15 mm, heat is confined in the tubular structure 400 sometimes.

(3-2) Manufacturing Method of Tubular Structure for Fixing Particulate Matter Detection Device:

There is not any special restriction on the manufacturing method of the tubular structure for fixing the particulate matter detection device, but the tubular structure for fixing the particulate matter detection device shown in, for example, FIGS. 4 and 5 can be manufactured by a method as follows.

To prepare the first holding tube, a stainless steel tube (the stainless tube) having predetermined diameter, length and thickness is prepared, and the end thereof corresponding to the first end is processed into a thread. It is to be noted that a portion corresponding to the bottom portion (the bottom portion of the tubular structure) 106 of the particulate matter detection device fixing tubular structure 400 shown in FIGS. 4 and 5 is preferably formed by cutting the portion integrally with the tube. Moreover, the portion corresponding to the bottom portion (the bottom portion of the tubular structure) 106 is preferably provided with a through hole through which the particulate matter detection device is passed (the hole through which the portion corresponding to the bottom portion passes). Then, the flange portion having a hexagonal outer shape is attached to the stainless tube by welding. Conditions such as the shape and size of the stainless tube are preferably set so as to obtain a preferable tubular structure for fixing the particulate matter detection device described above.

When the second holding tube is prepared, a stainless steel tube (the stainless tube) having predetermined diameter, length and thickness is prepared, and both ends are processed so as to decrease the diameters thereof. As shown in FIGS. 4 and 5, the outer shape of one end is formed into a truncated conical shape, and the outer shape of the other end is formed into a shape of a cylinder connected onto a truncated cone. Conditions such as the shape and size of the stainless tube are preferably set so as to obtain a preferable tubular structure for fixing the particulate matter detection device in the above embodiment of the present invention.

The stainless tube processed for preparing the first holding tube and the stainless tube processed for preparing the second holding tube are bonded by welding one end of the second holding tube to the end of the first holding tube which is not processed into a thread, whereby the tubular structure for fixing the particulate matter detection device as shown in FIGS. 4 and 5 is preferably obtained. As a method for welding the first holding tube to the second holding tube, laser welding, Tig welding or the like is preferable. It is to be noted that a plug may be made of a predetermined rubber.

(3-3) Tubular Structure for Fixing Particulate Matter Detection Device Provided with Protective Equipment:

As shown in FIGS. 4 and 5, the tubular structure for fixing the particulate matter detection device provided with the protective equipment of the present embodiment includes the tubular first holding tube 101 having the fixing structure portion 104 at the first end 102 as one end thereof, and the tubular second holding tube 111 having one end 112 fixed to the second end 103 as the other end of the first holding tube 101 so that the second holding tube is coaxial with the first holding tube 101. The embodiment (the particulate matter detection device protective equipment 100) of the protective equipment for the particulate matter detection device of the present invention is detachably attached to a tip 107 of the first holding tube 101. Moreover, the particulate matter detection device 121 made of the ceramic material, prolonged in one direction and having the detecting portion 122 of the particulate matter at one end thereof and the takeout portion 123 of the wiring line at the other end thereof is disposed so that the detecting portion 122 projects to the outside from the first end 102 of the first holding tube 101 and so that the takeout portion 123 of the wiring line is positioned in the second holding tube 111, and the particulate matter detection device is fixed to the pipe of the exhaust gas by the fixing structure portion 104 of the first holding tube 101 so that the detecting portion 122 of the particulate matter detection device 121 is positioned in the pipe of the exhaust gas. In this way, the tubular structure for fixing the particulate matter detection device provided with the protective equipment is provided with the particulate matter detection device and fixed to the pipe of the exhaust gas, whereby the particulate matter detection device is protected by the protective equipment 100, and the particulate matter detection device can be prevented from being cooled by the flow of the exhaust gas or the like in the pipe. Furthermore, water mixed with the exhaust gas or the like can be prevented from adhering to the particulate matter detection device, which can prevent the particulate matter detection device heated to a high temperature from being broken down by the water.

When the protective equipment 100 is attached to the tip 107 of the first holding tube of the particulate matter detection device fixing tubular structure 400, the fitting portion 11 of the protective equipment 100 is preferably fitted into and fixed to the tip 107 of the first holding tube of the particulate matter detection device fixing tubular structure 400. Moreover, a concave portion is formed in a side surface portion of the tip 107 of the first holding tube of the particulate matter detection device fixing tubular structure 400, and the projection 12 formed in the fitting portion 11 of the protective equipment 100 is fitted into the concave portion, whereby the protective equipment 100 can preferably detachably be attached to the particulate matter detection device fixing tubular structure 400. As shown in FIGS. 4 and 5, the tip 107 of the first holding tube of the particulate matter detection device fixing tubular structure 400 preferably has such a shape that the tip comes in contact with and is received in the fitting portion 11 of the protective equipment 100. The length of the tip 107 of the first holding tube of the particulate matter detection device fixing tubular structure 400 in the central axis direction thereof is preferably approximately equal to that of the inner periphery of the fitting portion 11 of the protective equipment 100 in the central axis direction. Moreover, in a case where the protective equipment 100 is attached to the particulate matter detection device fixing tubular structure 400 to which the particulate matter detection device is attached, the protective equipment 100 preferably does not come in contact with the particulate matter detection device. If they come in contact, the heat of the particulate matter detection device is taken by the protective equipment 100 sometimes.

The outer diameter of the fitting portion 11 of the protective equipment 100 is preferably smaller than the diameter of a thread structure portion as the fixing structure portion 104 of the particulate matter detection device fixing tubular structure 400 excluding thread ridges. In consequence, the internal thread corresponding to the thread structure (the external thread) of the fixing structure portion 104 of the particulate matter detection device fixing tubular structure 400 is formed in the pipe. When the tubular structure 500 for fixing the particulate matter detection device provided with the protective equipment is fixed to the internal thread, the protective equipment 100 can easily be inserted into the hole of the internal thread formed in the pipe.

In a case where the tubular structure for fixing the particulate matter detection device provided with the protective equipment of the present embodiment is fixed to the pipe of the exhaust gas while the particulate matter detection device is attached to the tubular structure, an angle between a direction opposite to an exhaust gas flowing direction in the pipe and a direction from the gas discharge port 6 of the protective equipment 100 to the gas introduction port 5 thereof is preferably from 45 to 75°, further preferably 50 to 70°, especially preferably 55 to 65°, most preferably 60°. The sensitivity of the inspection of the particulate matter by the particulate matter detection device improves, as the angle gets closer to 60°.

Another embodiment (the particulate matter detection device protective equipment 200) or still another embodiment (the particulate matter detection device protective equipment 300) of the protective equipment for the particulate matter detection device of the present invention is attached to the particulate matter detection device fixing tubular structure 400. This configuration is also a preferable embodiment of the tubular structure for fixing the particulate matter detection device provided with the protective equipment of the present invention.

(4) Particulate Matter Detection Device:

The particulate matter detection device fixed to the pipe through which the exhaust gas flows by the tubular structure for fixing the particulate matter detection device provided with the protective equipment of the present invention is a particulate matter detection device made of a ceramic material, prolonged in one direction and having a detecting portion of a particulate matter at one end thereof and a takeout portion of a wiring line at the other end thereof. Examples of the particulate matter detection device include a particulate matter detection device described in Japanese Patent Application No. 2008-246461, and specifically include the plate-like particulate matter detection device 121 made of a ceramic material and shown in FIGS. 6A and 6B. The particulate matter detection device 121 has a detecting portion 122 at one end 165 of a detection device main body 161, and the detecting portion 122 includes a through hole (the through hole of the detection device main body) 162 and a pair of electrodes embedded so as to sandwich the through hole 162 therebetween. Moreover, a voltage is applied across the pair of electrodes, a particulate matter in an exhaust gas which has flowed into the through hole 162 is electrically caused to adhere to the inner wall surface of the through hole or the like, and the impedance or the like of the inner wall surface of the through hole is measured to detect the adhering amount of the particulate matter or the like. Moreover, the particulate matter detection device 121 includes a heater for temperature control.

In the particulate matter detection device 121, at an end (the other end) 166 of the device opposite to the through hole 162, a takeout terminal 163 connected to one of the pair of electrodes is disposed, and a takeout terminal 164 connected to the other electrode of the pair of electrodes is disposed on the surface of a position between the one end of the device and the other end thereof. A portion provided with the takeout terminal 163 is a takeout portion 123. The takeout terminals 163, 164 are portions connected to an external electric wiring line. In such a particulate matter detection device, the detecting portion 122 is directly inserted into a high-temperature pipe to measure the particulate matter, and hence the detecting portion 122 is disposed away from the takeout portion 123 so that the takeout portion 123 vulnerable to heat does not have the high temperature. Therefore, the particulate matter detection device is prolonged in one direction, and includes the detecting portion 122 disposed at one end of the device and the takeout portion 123 disposed at the other end thereof, which prevents the heat on a detecting portion 122 side from being easily conducted to a takeout portion 123 side.

The material of the particulate matter detection device is preferably at least one selected from the group consisting of alumina, cordierite, mullite, glass, zirconia, magnesia and titania. Moreover, cordierite is further preferable, because it is excellent in resistance to thermal shock. Furthermore, the length of the particulate matter detection device is preferably from 70 to 130 mm, the thickness thereof is preferably from 0.5 to 3 mm, and the width thereof (the length thereof in a direction in which a gas flows in the detecting portion) is preferably from 2 to 20 mm. Moreover, examples of the material of the takeout terminals 163, 164 include nickel, platinum, chromium, tungsten, molybdenum, aluminum, gold, silver, copper, stainless steel and Kovar.

### EXAMPLES

Hereinafter, the present invention will further specifically be described with respect to examples, but the present invention is not limited to these examples.

### Example 1

A tubular structure for fixing a particulate matter detection device provided with a protective equipment, having a shape shown in FIGS. 4 and 5, was prepared. The preparation of the tubular structure for fixing the particulate matter detection device was performed as follows. First, a stainless tube having a diameter (the outer diameter) of 14 mm, a length of 68 mm and a thickness of 0.5 mm was prepared, and the end of the tube corresponding to a first end was processed into a thread. Moreover, a portion of the tube corresponding to a bottom portion was formed by cutting the portion integrally with the tube. In the bottom portion, a rectangular through hole of 7.1×12.1 mm was formed. Moreover, a flange portion having a hexagonal outer shape was attached to a position adjacent to the position where the thread portion of the stainless tube was formed by welding, and the stainless tube processed for preparing a first holding tube was obtained.

Next, a stainless tube having a diameter (the outer diameter) of 20 mm, a length of 47 mm and a thickness of 0.5 mm was prepared. Then, both the ends of the prepared stainless tube were processed so as to decrease the diameters thereof, and as shown in FIGS. 4 and 5, the outer shape of one end was formed into a truncated conical shape, and the outer shape of the other end was formed into a shape of a cylinder connected onto a truncated cone, whereby the stainless tube processed for preparing a second holding tube was obtained. As to the size of the cross section of the truncated conical end cut along a plane including the central axis, an upper bottom had a size of 16.5 mm, a lower bottom had a size of 20 mm, and a height was 3 mm. Moreover, as the size of the end having a shape of the cylinder connected onto a truncated cone, in a truncated conical portion, the size of an upper bottom thereof was 14 mm, the size of a lower bottom thereof was 20 mm and a height thereof was 3 mm, and in a cylindrical portion, the diameter of the bottom surface thereof was 20 mm and a height thereof was 15 mm.

Next, the stainless tube processed for preparing the first holding tube and the stainless tube processed for preparing the second holding tube were bonded by laser-welding one end of the second holding tube to the end of the first holding tube which was not processed into a thread, whereby the tubular structure for fixing the particulate matter detection device was obtained as shown in FIGS. 4 and 5. The length of the first holding tube inserted into the second holding tube was 8 mm.

A protective equipment for a particulate matter detection device similar to the particulate matter detection device protective equipment 100 shown in FIGS. 1A to 1D was prepared. A trunk portion was prepared by pressing austenitic stainless steel having a thickness of 0.5 mm. The shape of the trunk portion was a cylindrical shape having a bottom surface diameter of 15 mm and a length of 43 mm. When the trunk portion was prepared by a press, a gas introduction port, a gas discharge port and a through hole were cut by the press. The length of each of the gas introduction port and the gas discharge port in the central axis direction of the trunk portion was 8 mm, and the length thereof in a peripheral direction was 3 mm. The gas introduction port and the gas discharge port were positioned so that they face each other via the central axis of the trunk portion (in the cross section of the trunk portion crossing the central axis thereof at right angles, the gas introduction port, the gas discharge port and the central axis of the trunk portion were linearly arranged). The through hole had a round shape having a diameter of 3 mm, and was formed in the center of the bottom portion of the protective equipment main body. Inlet side guide plates and outlet side guide plates were prepared, and bonded to contour portions of the gas introduction port of the trunk portion by laser welding as shown in FIGS. 1A and 1C. Each of the inlet side guide plates and the outlet side guide plates was made of austenitic stainless steel and had a thickness of 0.5 mm, a length of 8 mm in a longitudinal direction and a length of 5 mm in a direction crossing the longitudinal direction at right angles. A fitting portion was prepared by pressing austenitic stainless steel having a thickness of 0.5 mm. The shape of the fitting portion was a cylindrical shape having a bottom surface diameter of 15 mm and a length of 41 mm. A projection was formed on the inner surface of the fitting portion by cutting simultaneously with the preparation of the fitting portion. The trunk portion was bonded to the fitting portion by laser welding.

A protective equipment for the particulate matter detection device was attached to the obtained tubular structure for fixing the particulate matter detection device, to obtain the tubular structure for fixing the particulate matter detection device provided with the protective equipment. It is to be noted that when “a heat release property test” and “a crack generation test” are performed as follows, the particulate matter detection device needs to be attached to the inside of the tubular structure for fixing the particulate matter detection device. After attaching the particulate matter detection device to the inside of the stainless tube processed for preparing the first holding tube, the stainless tube for preparing the first holding tube and the stainless tube for preparing the second holding tube were bonded. Moreover, a columnar rubber plug having a bottom surface diameter of 13 mm and a height of 15 mm was prepared, and a through hole for passing a wiring line therethrough was formed. As the material of the plug, silicon rubber was used.

The obtained tubular structure for fixing the particulate matter detection device provided with the protective equipment was subjected to a test (the heat release property test) for confirming the heat release (cooling) state of the particulate matter detection device and a test (the crack generation test) for confirming the crack generation state of the particulate matter detection device by the following methods. Results are shown in Table 1.

(Heat Release Property Test)

There is prepared a pseudo stainless exhaust pipe having a 50 A size and provided with a socket to which the tubular structure for fixing the particulate matter detection device provided with the protective equipment (or the tubular structure for fixing the particulate matter detection device which is not provided with the protective equipment) can be attached, and the pseudo stainless exhaust pipe is disposed in parallel with the floor surface so as to dispose the socket on the upside. The tubular structure for fixing the particulate matter detection device provided with the protective equipment is attached to the socket so that a gas inlet of a gas introduction pipe faces the upstream side. Behind (on the downstream side of) the pseudo stainless exhaust pipe, an electromotive fan capable of acquiring a flow of 1.5 m³/minute is attached. In this case, all air generated by the electromotive fan flows through the pseudo stainless exhaust pipe, and the flow of the air is not formed outside the pseudo stainless exhaust pipe. Moreover, the direction of the flow of the air by the electromotive fan is a direction from the tubular structure for fixing the particulate matter detection device provided with the protective equipment to the electromotive fan. While the flow of the air is not present, a voltage is applied to the particulate matter detection device so that a heater portion has a temperature of 700° C. In this case, a heater power (a voltage value, a current value) and a resistance value are simultaneously measured, and temperature (resistance value temperature) is calculated from the resistance value. The application of the voltage is stopped once, it is confirmed that the temperature of the heater portion lowers approximately to room temperature, and the electromotive fan is operated. The voltage is applied again to regulate the heater power (the voltage value, the current value) so that the temperature of the heater portion of the particulate matter detection device becomes 700° C. At this time, the resistance value temperature is measured. The heater power and the resistance value temperature are measured, when a stationary state is obtained, after kept for five minutes and stabilized. In consequence, 20% or more increase of the heater power with the flow of the air as compared with the heater power without the flow of the air is judged as a failure (x). The increase of less than 20% is judged to be successful (o).

(Crack Generation Test)

There is prepared the pseudo stainless exhaust pipe having a 50 A size and provided with the socket to which the tubular structure for fixing the particulate matter detection device provided with the protective equipment (or the tubular structure for fixing the particulate matter detection device which is not provided with the protective equipment) can be attached, and the pseudo stainless exhaust pipe is disposed in parallel with the floor surface so as to dispose the socket on the upside. A heater resistance value and a capacitance value of the particulate matter detection device are beforehand measured at room temperature. They are obtained as “initial numeric values”. The tubular structure for fixing the particulate matter detection device provided with the protective equipment is attached to the socket. Behind (on the downstream side of) the pseudo stainless exhaust pipe, the electromotive fan capable of acquiring a flow of 1.5 m³/minute is attached. In this case, all the air generated by the electromotive fan flows through the pseudo stainless exhaust pipe, and the flow of the air is not formed outside the pseudo stainless exhaust pipe. Moreover, the direction of the flow of the air by the electromotive fan is the direction from the tubular structure for fixing the particulate matter detection device provided with the protective equipment to the electromotive fan. Furthermore, on the upstream side of the tubular structure for fixing the particulate matter detection device provided with the protective equipment, an atomizer capable of atomizing water at a constant ratio per hour is attached. The atomization is performed at a ratio of 5 cm³ per second for five seconds, and afterward the atomization is not performed for 25 seconds. This is one cycle.

A test procedure is as follows. While the flow of the air is not present, the voltage is applied to the particulate matter detection device so that the heater portion has a temperature of 700° C. In this case, the heater power (the voltage value, the current value) and the resistance value are simultaneously measured, and the temperature (the resistance value temperature) is calculated from the resistance value. The application of the voltage is stopped once, it is confirmed that the temperature of the heater portion lowers approximately to room temperature, and the electromotive fan is operated. The voltage is applied again to regulate the heater power (the voltage value, the current value) so that the temperature of the heater portion becomes 700° C. At this time, the resistance value temperature is measured. The heater power and the resistance value temperature are measured, when the stationary state is obtained, after kept for five minutes and stabilized. Afterward, in the above stationary state (700° C.), the water is atomized as much as ten cycles, and the application of the voltage is stopped. When the temperature of the heater portion lowers approximately to room temperature, the heater resistance value and the capacitance value are measured. In consequence, a case where at least one of the heater resistance value and the capacitance value deviates from a range of ±10% of “the initial numeric value” (excluding ±10%) is judged as a failure (x). Moreover, a case where “an abnormality is present” in a red check is judged as a failure (x). A case where the value does not deviate from a range of ±10% of “the initial numeric value” (including ±10%) and “the abnormality is not present” also in the red check is judged to be successful (o).

Here, the red check is a test in which the particulate matter detection device is immersed into a solution (trade name: Neo Glow (F-4A-C) manufactured by Eishin Kagaku Co., Ltd.) of hydrogen carbide oil blended with a plastic solvent, a surfactant and a fluorescence dyestuff (red), left to stand for a few seconds and lightly washed with water, and then the presence of a portion dyed in red is confirmed. A cracked portion is dyed in red. Moreover, when any red dyed portion is not present, it is judged that “any abnormality is not present”. When the red dyed portion is present, it is judged that “the abnormality is present”.

As the particulate matter detection device for a strength test and the heat release property test, the ceramic plate-like particulate matter detection device 121 shown in FIGS. 6A and 6B was used. The particulate matter detection device 121 included a detecting portion 122 at one end 165 of a detection device main body 161, and the detecting portion 122 included a through hole (the through hole of the detection device main body) 162 and a pair of electrodes embedded so as to sandwich the through hole 162 therebetween. Moreover, the voltage was applied across the pair of electrodes, and the particulate matter in an exhaust gas which had flowed into the through hole 162 was electrically caused to adhere to the inner wall surface of the through hole, whereby the impedance of the inner wall surface of the through hole or the like was measured to detect the amount of the adhering particulate matter or the like. Moreover, the particulate matter detection device 121 included a heater portion for temperature control. The material of the particulate matter detection device was cordierite. Furthermore, the particulate matter detection device had a length of 116 mm, a thickness of 1.75 mm and a width (the length in a direction in which the gas flowed in the detecting portion) of 7 mm. The material of portions such as the electrodes across which the voltage was applied was tungsten.

TABLE 1

| | Heat release | |
|---|---|---|
| | property test | Crack generation test |
| Example 1 | ∘ | ∘ |
| Reference example | x | x |

### Reference Example

In the heat release property test and the crack generation test, a particulate matter detection device was attached to a tubular structure for fixing the particulate matter detection device, and a protective equipment for the particulate matter detection device was not attached, whereby this tubular structure was prepared as a reference example. The tubular structure for fixing the particulate matter detection device and the particulate matter detection device were prepared in the same manner as in Example 1. In the same manner as in Example 1, the heat release property test and the crack generation test were performed. Results are shown in Table 1.

It is seen from Table 1 that the tubular structure for fixing the particulate matter detection device provided with the protective equipment does not easily release heat, and is not easily cracked.

The present invention can preferably be utilized for fixing the particulate matter detection device to an exhaust pipe or the like of a car engine or the like.

### DESCRIPTION OF REFERENCE NUMERALS

1: trunk portion, 2: one end, 3: bottom portion (the bottom portion of a protective equipment main body), 3a: through hole (the through hole of the bottom portion of the protective equipment main body), 4: protective equipment main body, 5: gas introduction port, 5a: opening, 5b: contour portion of gas introduction port, 6: gas discharge port, 7: other end, 7a: opening side end, 11: fitting portion, 12: projection, 21: inlet side guide plate, 22: outlet side guide plate, 23: squeezed portion, 24: exhaust gas flow, 25: connecting portion, 31: gas introduction tube, 32: hollow portion, 33: gas inlet, 101: first holding tube, 102: first end, 103: second end, 104: fixing structure portion, 105: flange portion, 106: bottom portion (the bottom portion of the tubular structure), 107: tip of first holding tube, 111: second holding tube, 112: one end, 113: middle portion, 114: plug, 121: particulate matter detection device, 122: detecting portion, 123: takeout portion, 124: contact member, 161: detection device main body, 162: through hole (the through hole of the detection device main body), 163, 164: takeout terminal, 165: one end, 166: other end, 100, 200 and 300: protective equipment for particulate matter detection device, 400: particulate matter detection device fixing tubular structure, and 500: tubular structure for fixing particulate matter detection device provided with protective equipment.

What is claimed is:

1. A protective equipment for a particulate matter detection device comprising:
a bottomed cylindrical protective equipment main body having a cylindrical trunk portion and a bottom portion which closes one end of the trunk portion,
the protective equipment main body being provided with a gas introduction port which extends through a wall of the trunk portion and through which a gas flows from the outside to the inside, and a gas discharge port which extends through the wall of the trunk portion at a position facing the gas introduction port and through which the gas is discharged from the inside to the outside.

2. The protective equipment for the particulate matter detection device according to claim 1, further comprising:
plate-like inlet side guide plates formed so as to cross the gas introduction port at right angles and so as to extend from at least a part of a contour portion of the gas introduction port to the inside of the trunk portion.

3. The protective equipment for the particulate matter detection device according to claim 1, wherein the trunk portion includes a squeezed portion whose cross section crossing a central axis at right angles has a sectional area smaller than that of each of the other portions, and the squeezed portion is provided with the gas introduction port and the gas discharge port.

4. The protective equipment for the particulate matter detection device according to claim 1, further comprising:
a gas introduction tube disposed outside the trunk portion so that the gas introduction port is connected to a hollow portion.

5. The protective equipment for the particulate matter detection device according to claim 1, wherein a through hole is formed in the bottom portion of the protective equipment main body.

6. A tubular structure for fixing a particulate matter detection device provided with a protective equipment, comprising: a tubular first holding tube having a fixing structure portion at a first end as one end thereof, and a tubular second holding tube having one end fixed to a second end as the other end of the first holding tube so that the second holding tube is coaxial with the first holding tube,
wherein at the tip of the first holding tube, the protective equipment for the particulate matter detection device according to claim 1 is detachably attached;
the particulate matter detection device made of a ceramic material, prolonged in one direction and having a detecting portion of a particulate matter at one end thereof and a takeout portion of a wiring line at the other end thereof is disposed so that the detecting portion projects to the outside from the first end of the first holding tube and so that the takeout portion of the wiring line is positioned in the second holding tube; and
the particulate matter detection device is fixed to a pipe of an exhaust gas by the fixing structure portion of the first holding tube so that the detecting portion of the particulate matter detection device is positioned in the pipe of the exhaust gas.

7. The protective equipment for the particulate matter detection device according to claim 2, wherein the trunk portion includes a squeezed portion whose cross section crossing a central axis at right angles has a sectional area smaller than that of each of the other portions, and the squeezed portion is provided with the gas introduction port and the gas discharge port.

8. The protective equipment for the particulate matter detection device according to claim 2, further comprising:
a gas introduction tube disposed outside the trunk portion so that the gas introduction port is connected to a hollow portion.

9. The protective equipment for the particulate matter detection device according to claim 3, further comprising:
a gas introduction tube disposed outside the trunk portion so that the gas introduction port is connected to a hollow portion.

10. The protective equipment for the particulate matter detection device according to claim 7, further comprising:
a gas introduction tube disposed outside the trunk portion so that the gas introduction port is connected to a hollow portion.

11. The protective equipment for the particulate matter detection device according to claim 2, wherein a through hole is formed in the bottom portion of the protective equipment main body.

12. The protective equipment for the particulate matter detection device according to claim 3, wherein a through hole is formed in the bottom portion of the protective equipment main body.

13. The protective equipment for the particulate matter detection device according to claim 7, wherein a through hole is formed in the bottom portion of the protective equipment main body.

14. A tubular structure for fixing a particulate matter detection device provided with a protective equipment, comprising: a tubular first holding tube having a fixing structure portion at a first end as one end thereof, and a tubular second holding tube having one end fixed to a second end as the other end of the first holding tube so that the second holding tube is coaxial with the first holding tube,
wherein at the tip of the first holding tube, the protective equipment for the particulate matter detection device according to claim 2 is detachably attached;
the particulate matter detection device made of a ceramic material, prolonged in one direction and having a detecting portion of a particulate matter at one end thereof and a takeout portion of a wiring line at the other end thereof is disposed so that the detecting portion projects to the outside from the first end of the first holding tube and so that the takeout portion of the wiring line is positioned in the second holding tube; and
the particulate matter detection device is fixed to a pipe of an exhaust gas by the fixing structure portion of the first holding tube so that the detecting portion of the particulate matter detection device is positioned in the pipe of the exhaust gas.

15. A tubular structure for fixing a particulate matter detection device provided with a protective equipment, comprising: a tubular first holding tube having a fixing structure portion at a first end as one end thereof, and a tubular second holding tube having one end fixed to a second end as the other end of the first holding tube so that the second holding tube is coaxial with the first holding tube,
wherein at the tip of the first holding tube, the protective equipment for the particulate matter detection device according to claim 3 is detachably attached;
the particulate matter detection device made of a ceramic material, prolonged in one direction and having a detecting portion of a particulate matter at one end thereof and a takeout portion of a wiring line at the other end thereof is disposed so that the detecting portion projects to the outside from the first end of the first holding tube and so that the takeout portion of the wiring line is positioned in the second holding tube; and
the particulate matter detection device is fixed to a pipe of an exhaust gas by the fixing structure portion of the first holding tube so that the detecting portion of the particulate matter detection device is positioned in the pipe of the exhaust gas.

16. A tubular structure for fixing a particulate matter detection device provided with a protective equipment, comprising: a tubular first holding tube having a fixing structure portion at a first end as one end thereof, and a tubular second holding tube having one end fixed to a second end as the other end of the first holding tube so that the second holding tube is coaxial with the first holding tube,
wherein at the tip of the first holding tube, the protective equipment for the particulate matter detection device according to claim 4 is detachably attached;
the particulate matter detection device made of a ceramic material, prolonged in one direction and having a detecting portion of a particulate matter at one end thereof and a takeout portion of a wiring line at the other end thereof is disposed so that the detecting portion projects to the outside from the first end of the first holding tube and so that the takeout portion of the wiring line is positioned in the second holding tube; and
the particulate matter detection device is fixed to a pipe of an exhaust gas by the fixing structure portion of the first holding tube so that the detecting portion of the particulate matter detection device is positioned in the pipe of the exhaust gas.

17. A tubular structure for fixing a particulate matter detection device provided with a protective equipment, comprising: a tubular first holding tube having a fixing structure portion at a first end as one end thereof, and a tubular second holding tube having one end fixed to a second end as the other end of the first holding tube so that the second holding tube is coaxial with the first holding tube,
wherein at the tip of the first holding tube, the protective equipment for the particulate matter detection device according to claim 5 is detachably attached;
the particulate matter detection device made of a ceramic material, prolonged in one direction and having a detecting portion of a particulate matter at one end thereof and a takeout portion of a wiring line at the other end thereof is disposed so that the detecting portion projects to the outside from the first end of the first holding tube and so that the takeout portion of the wiring line is positioned in the second holding tube; and
the particulate matter detection device is fixed to a pipe of an exhaust gas by the fixing structure portion of the first holding tube so that the detecting portion of the particulate matter detection device is positioned in the pipe of the exhaust gas.
